SEQUENCE LISTING (2 sequences)

SEQ ID NO: 1 - DNA, 21 bp, unidentified
  source 1..21: mol_type = unassigned DNA
ccgagtttgatcmtggctcag

SEQ ID NO: 2 - DNA, 18 bp, unidentified
  source 1..18: mol_type = unassigned DNA
gctgcctcccgtaggagt

(19) United States
(12) Patent Application Publication
(10) Pub. No.: US 2025/0290158 A1
(43) Pub. Date: Sep. 18, 2025

(54) METHOD FOR ASSESSING SKIN

(71) Applicant: CONOPCO INC., D/B/A UNILEVER, Ciiffs, NJ (US)

(72) Inventors: Luonan CHEN, Shanghai (CN); Chung-Ching CHU, Shanghai (CN); Mingming PU, Shanghai (CN); Lu WANG, Tianjin (CN); Yining XU, Shanghai (CN)

(73) Assignee: CONOPCO INC., D/B/A UNILEVER, Englewood Cliffs, NJ (US)

(21) Appl. No.: 18/576,067

(22) PCT Filed: Jul. 1, 2022
(86) PCT No.: PCT/EP2022/068283

§ 371 Date: Jan. 2, 2024

(30) Foreign Application Priority Data
Jul. 5, 2021 (WO) PCT/CN2021/104588
Sep. 2, 2021 (EP) 21194475.6

(51) Int. Cl. C12Q 1/689; C12Q 1/6874

(52) U.S. Cl. CPC C12Q 1/689; C12Q 1/6874

(57) ABSTRACT

Disclosed is a method of assessing skin comprising the steps of: A) identifying a first individual microbiome network of the desired skin surface of a person in need of skin assessment; B) comparing the first individual microbiome network with a benchmark individual microbiome network; and C) providing a cosmetic composition to change the first individual microbiome network toward the benchmark individual microbiome network.

### FIELD OF THE INVENTION

The present invention relates to a method of assessing skin.

### BACKGROUND OF THE INVENTION

The skin is the largest organ of human body. It protects our body from external factors such as the environment, pollution, and etc. Our skin harbours a diverse range of microorganisms, which live as a community and function as a component of skin biology. It is believed that the microbiome plays at least a partial role in a number of skin conditions. The diversity, composition, and stability of skin microbiomes may be influenced by host intrinsic factors for example genetics, or environmental factors.

Network theory has been employed as a promising approach to analyze the complex skin microbiome with multifaceted interactions between microbiota. The microbiome network analysis is usually conducted to compare two or more sets of individuals and therefore limited to study the mean value across a population of samples or subjects. The skin microbiomes of a specific individual in a community cannot be reflected by current network analysis.

However, there is an increasing need for provide personalized cosmetic compositions tailored to meet the frequently changing requirements of a particular user. Therefore, the present inventors developed a method for assessing skin comprising the steps of identifying a first individual microbiome network. By such method, a specific person may be given a personalized cosmetic composition according to the individual microbiome network of such person.

### SUMMARY OF THE INVENTION

In a first aspect, the present invention is directed to a method of assessing skin comprising the steps of: A) identifying a first individual microbiome network of the desired skin surface of a person in need of skin assessment; B) comparing the first individual microbiome network with a benchmark individual microbiome network; and C) providing a cosmetic composition to change the first individual microbiome network toward the benchmark individual microbiome network, wherein identifying a first individual microbiome network comprises the steps of: (a) obtaining a first population microbiome network from a group of samples consisting of N individuals including said person, wherein N is an integer at least 20; (b) obtaining a second populational microbiome network from the same group of samples without the sample from said person; and (c) determining the first individual microbiome network of said person by subtracting the effects of the second population microbiome network from the first population microbiome network.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

“Microbiome” as used herein refers to the diverse ecological community of commensal bacteria, fungi, viruses, and/or parasites that are associated with an organism.

“Microbiome network” as used herein refers to a co-occurrence network built through network theory of microbiome abundance data, indicating the direct interactions and/or indirect interactions among the microbiomes.

“Population Microbiome network” as used herein refers to the microbiome network of a group of samples, which typically reflects the interactions among the group of samples. Population microbiome network is exchangeable with microbiome network of population which may be abbreviated as MNP.

“Individual microbiome network” as used herein refers to the microbiome network of a single sample. Individual microbiome network is exchangeable with microbiome network of individual which may be abbreviated as MNI.

To fully understand the preferred embodiment of the present invention, the network analysis of microbiome will be described. Network models can portray the members of a microbial community along with inference about their interactions. The microbiome network is usually visualized by a set of nodes connected to each other by many edges. “Nodes” as used herein refer to individual entities that are the building blocks of a microbiome network, typically represents a microbiome taxonomy feature such as an amplicon sequence variant (ASV), operational taxonomic unit (OTU), a microbial species, or a microbial genus. “Edges”, as used herein refers to the connections between nodes in a network, which reflect the association, relation, and interaction between the nodes. “Node degree” as used herein refers to the number of edges between itself and other nodes. The node degree is usually used to describe the connectivity of the network. The frequency distribution of node degree is usually used to infer network robustness. “Clustering coefficient” as used herein refers to ratio of the number of edges between the neighbors of a node, and the maximum number of edges that could possibly exist between its neighbors. The clustering coefficient of a node is always a number between 0 and 1.

The way of identifying the first individual microbiome network comprises the steps of: (a) obtaining a first microbiome network of population (MNP) from a group of samples consisting of N individuals including said person, wherein N is an integer at least 3; (b) obtaining a second MNP from the same group of samples without the sample from said person, i.e. N-1 samples; and (c) determining the microbiome network of said person by subtracting the effects of the second MNP from the first MNP. Preferably, N is an integer of at least 10, more preferably at least 20, even more preferably 35 to 1,000,000, and most preferably 50 to 100,000.

Preferably, the method of obtaining an MNP comprises the step of determining microbiome composition data of the desired skin surface; and generating an MNP by representing microbial organisms in each matrix as a network of plurality of nodes corresponding the group of samples.

Preferably, the way of determining microbiome composition data comprises extracting microbial DNA samples from the desired skin surfaces of a group of persons, preferably by tape stripping, swabbing, or buffer scrubbing, or any other methods suitable for body surface microbe collection; extracting DNA using any established methods for each sample; and sequencing of DNA samples by a sequencer to generate a plurality of DNA sequences. Preferably, the way of determining microbiome composition data further comprises the step of creating a matrix of microbial abundance profile of the operational taxonomic unit (OTU), amplicon sequence variant (ASV) or further accumulating at different taxonomy level corresponding to each person.

The microbiome network of population (MNP) may be generated in any suitable way. However, it is preferable that MNPs are constructed by MENA (Molecular Ecological Network Analysis), LSA (local similarity analysis), SparCC (Sparse Correlations for Compositional data) and NetCoMi (Network Construction and comparison for Microbiome data). It is preferable that MNPs are constructed by SParse InversE Covariance estimation for Ecological Association Inference (SPIEC-EASI) analysis from the matrix of sequencing counts. Preferably, the method of obtaining a MNP further comprises the step of visualizing the relationships of a set of nodes with edges.

Preferably, the microbiome composition of the present invention comprises a set of taxa comprising at least one of: Marvinbryantia (genus), Erysipelotrichales (order), Erysipelotrichia (class), Bacteroidetes (phylum), Staphylococcus (genus), Staphylococcaceae (family), Bacillales (order), Actinobacteria (class), Firmicutes (phylum), Actinobacteria (phylum), and Propionibacterium (genus).

“Skin” as used herein is meant to include skin on the face, oral and body (e.g., neck, chest, back, arms, underarms, hands, legs, buttocks and scalp). The desired skin surface may be selected from any surface of body skin and/or face skin. Preferably, the desired skin surface is selected from scalp or face skin. Most preferably, the desired skin surface is selected from face skin, in particular cheek. It should be noted that the sample is from the same body site of said person and N individuals.

Preferably, step B) comprises the step of obtaining a benchmark individual microbiome network and comparing the obtained benchmark individual microbiome network with the first individual microbiome network. The benchmark individual microbiome network refers to individual microbiome network of benchmark skin surface. The benchmark skin surface refers to skin surface at the substantially same position as the desired skin surface but having healthy skin conditions. The benchmark persons refer to a group of persons having healthy skin conditions at the substantially same position as the desired skin surface in a sufficient number (at least 3, preferably 15 to 10,000).

The benchmark individual microbiome network may be obtained in any suitable way. However, it is preferred that the benchmark individual microbiome network is obtained by selecting a group of benchmark persons and obtaining an averaged benchmark individual microbiome network from benchmark skin surface of the group of benchmark persons. Alternatively, or additionally, the step of identifying a benchmark individual microbiome network comprising the step of finding a benchmark individual microbiome network from a first database. The first database contains information of benchmark individual microbiome networks of different skin surface. Preferably, this first database is formed through testing on a wide range of skin surfaces of individuals, so as to produce a library in which the benchmark individual microbiome networks are contained.

Preferably, step (B) comprises identifying a first shift of the first individual microbiome network from the benchmark individual microbiome network. Preferably, the shift comprises shift of at least one of attributes selected from node, edge, node degree, clustering coefficient, and visualized microbiome network. More preferably the shift comprises shift of node degree and/or visualized microbiome network. Preferably, the method further comprises a step of assessing the skin conditions of the desired skin surface and associating the first shift with the skin conditions.

Preferably, the skin conditions comprise skin barrier, skin moisture, aging, wrinkle, darkness, or a combination thereof.

“Cosmetic composition” refers to any product applied to a human body for improving appearance, sun protection, reducing wrinkled appearance or other signs of photoaging, odor control, skin lightening, even skin tone, or general aesthetics. Non-limiting examples of cosmetic compositions include lotions, creams, facial masks, gels, sticks, antiperspirants, deodorants, liquid or gel body washes, soap bars, oral care products, and sunless tanners.

The composition preferably comprises a surfactant. More than one surfactant may be included in the composition. The surfactant may be chosen from soap, non-soap anionic, cationic, non-ionic, amphoteric surfactant and mixtures thereof. Many suitable surface-active compounds are available and are fully described in the literature, for example, in “Surface-Active Agents and Detergents”, Volumes I and II, by Schwartz, Perry and Berch. The preferred surfactant that can be used are soaps, non-soap anionic, non-ionic surfactant, amphoteric surfactant or a mixture thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred.

Most preferred non-soap anionic surfactant are linear alkylbenzene sulphonate, particularly linear alkylbenzene sulphonates having an alkyl chain length of from C₈ to C₁₅. It is preferred if the level of linear alkylbenzene sulphonate is from 0 wt % to 30 wt %, more preferably from 1 wt % to 25 wt %, most preferably from 2 wt % to 15 wt %, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). It is preferred if the level of non-ionic surfactant is from 0 wt % to 30 wt %, preferably from 1 wt % to 25 wt %, most preferably from 2 wt % to 15 wt %, by weight of a fully composition.

Suitable amphoteric surfactants preferably are betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0 to 20 wt %, more preferably from 1 to 10 wt %, by weight of the composition.

Water-insoluble skin benefit agents may also be formulated into the compositions as conditioners and moisturizers. Examples include silicone oils; hydrocarbons such as liquid paraffins, petrolatum, microcrystalline wax, and mineral oil; and vegetable triglycerides such as sunflower seed and cottonseed oils.

The composition may comprise optional ingredients including pigment, moisturizing agent, organic sunscreen, skin glowing agent, fragrance, natural extract, or a combination thereof.

Pigments suitable for the present inventions are typically particles of refractive index materials greater than 1.3, more preferably greater than 1.8 and most preferably from 2.0 to 2.7. Examples of such pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof and most preferred pigment is titanium dioxide. The average diameter of the pigment is typical from 15 nm to 1 micron, more preferably from 35 nm to 800 nm, even more preferably from 50 nm to 500 nm and still even more preferably from 100 to 300 nm.

Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

Vitamin B3 compounds (including derivatives of vitamin B3) e.g. niacin, nicotinic acid or niacinamide are the preferred skin glowing agent as per the invention, most preferred being niacinamide.

Some compositions may include thickeners. These may be selected from cellulosics, natural gums and acrylic polymers but not limited by this thickening agent types. Amounts of thickener may range from 0.01 to 3% by weight of the active polymer (outside of solvent or water) in the compositions. Preservatives can desirably be incorporated into the compositions of this invention to protect against the growth of potentially harmful microorganisms.

Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatabilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

The composition may comprise water in amount of 10 to 95% by weight of the composition, more preferably from 25 to 90%, even more preferably from 32 to 85%, most preferably from 45 to 78% by weight of the composition.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹. Preferably, the composition is in the form of fluid.

Preferably, the step of providing a cosmetic composition comprises the step of topically applying the cosmetic composition onto the desired skin surface, preferably by human hand. The amount of the cosmetic composition is preferably 0.1 to 100 g, preferably 0.5 to 10 g for each time.

Preferably, the skin surface was treated by the cosmetic composition with a frequency of at least once a day, more preferably twice to four times a day. Preferably, such treatment continues for a duration of one week to one year, more preferably two weeks to three months.

Preferably, the cosmetic composition is provided according to a second database. Preferably, the second database comprises the information of the association of shift of MNIs and/or skin conditions with cosmetic compositions. This second database may be formed through testing on a wide range of individuals, so as to produce a library of association of shift of individual microbiome networks, and/or skin conditions with the cosmetic composition. Alternatively, or additionally, it will also be appreciated that the database may be obtained by virtue of mathematical correlations to obtain association of shift of MNIs, and/or skin conditions with the cosmetic composition. Thus, a suitable cosmetic composition may be provided.

Preferably, the method comprises the step of repeating (A), (B), and (C) until the identified first microbiome network is substantially same as the benchmark MNI.

The following examples are provided to facilitate an understanding of the invention. The examples are not intended to limit the scope of the claims.

### EXAMPLES

### Example 1

This example demonstrates the improvement of individual microbiome network by cosmetic composition.

### 1) Subject Selection

Two group of volunteers were recruited. The first group of 27 subjects has intense ageing appearance, and the second group of 35 subjects has normal ageing appearance (benchmark group). The individual microbiome networks for each subject in their group were tested and obtained.

### 2) Identification of Individual Microbiome Network (MNI)

The individual microbiome networks of the first group and the benchmark group were constructed by following below procedures.

Facial microbiome samples were collected from upper cheeks using a cup scrub technique with phosphate buffered saline buffer (pH 7.9) containing 0.1% TritonX-100 (93443, Sigma, Missouri, USA). Buffer samples were stored at −80° C. before analysis. As controls, mock community, blank buffer control and PCR negative control samples were included. Microbial DNA was extracted from the samples using a DNA extraction kit (DNeasy Blood & Tissue kit, 69506, Qiagen, Hilden, Germany) following the manufacturer's instructions.

The microbial DNAs were sequenced at the variable region (V1-V2) of the 16S rDNA gene for bacterial classification. The V1-V2 region was amplified using a primer set (forward primer (SEQ ID NO: 1): 5′-CCGAGTTTGATCMTGGCTCAG-3′ and reverse primer (SEQ ID NO: 2): 5′-GCTGCCTCCCGTAGGAGT-3′), and sequenced by Beijing Genomics Institute (BGI, Wuhan, China) by using fusion primers with dual indices and adapters. The quantity and quality of the libraries were analyzed by Bioanalyzer (Agilent Technologies, California, USA). Only qualified libraries were used for sequencing on the Illumina Miseq PE300 platform, initially resulting in approximately 159 million raw sequence paired reads. Sequences with low quality were discarded before analysis.

Microbiome composition was generated from clean sequencing raw data by QIIME (Quantitate Insights into Microbial Ecology) version 1.9.1. Taxonomy classification was carried out using a Lowest Common Ancestor methodology against the following databases: SILVA, NCBI, RDP, DDBJ, Greengenes, CAMERA, EMBL, EzTaxon. Finally, 143 million overlapping contigs were grouped into 729 OTUs. Microbiome composition of each sample were accumulated by sequencing counts according to taxonomy classification. Before microbiome network construction, OTUs that had frequencies of less than 80% in samples in each group were removed.

The individual microbiome network for each sample was obtained by follow procedures:

Step-1: input the sequencing data (relative abundances of species: x, y, z . . . ) with NV samples of p species. Let q=1. N is the number for all samples and q is the sequence number of a specific sample.

Step-2: calculate each partial correlation r_{xy,z}^{(N)} with/samples using SPIEC-EASI.

Step-3: calculate each partial correlation r_{xy,z}^{(N/q)} with N-1 samples by removing the qth sample using SPIEC-EASI.

Step-4: calculate qth sample's specific partial correlation for each pair of species x and y by following the equation:

$${l}_{xy , z}^{( q )} = {Nr}_{xy , z}^{( N )} - ( N - 1 ) {r}_{xy , z}^{( N / q )}$$

Step-5: Let q=q+1, and go to Step-3 until q=N. Partial correlations of all pairs for each sample form the individual microbiome network.

The node degrees (attribute to describe the connectivity of a network) for microbiome networks were for each group were calculated.

### 3) Cosmetic Composition Treatment to Alter the Individual Microbiome Network

By comparing the individual microbiome networks of the first group and the benchmark group, the subjects of the first group were provided a commercial facial cleanser and a commercial facial cream. Each subject applied the commercial facial cleanser and cream twice a day for four weeks. Then, the individual microbiome network of the subjects of the first group were constructed following the same procedure as described above. The node degrees for microbiome networks for each subject in the first group were calculated.

TABLE 1

| | Average node degree of MNIs |
|---|---|
| Second group (Benchmark group) | 2.76 |
| First group before product treatment | 1.91^{a} |
| First group after product treatment | 2.13^{b} |

^{a}Significant difference from second group (p < 0.05)
^{b}Significant difference from First group after product treatment (p < 0.05)

Table 1 indicates the average node degree of MNIs for the benchmark group and for the first group before and after product treatment. The node average degree for MNIs for the first group was significantly lower than that of benchmark group, indicating that the connectivity of MNIs for the first group before product treatment is much lower than that of benchmark group. It was further demonstrated that product intervention can significantly recover the lower node degree first group toward to the benchmark group, indicating that product intervention by suitable products is capable of providing healthier MNIs.

### Example 2

This example demonstrates the sensitivity of the method of assessing skin of the present invention.

### (a) Classification by Image Analysis

The VISIA CR® images (Canfield Scientific, Inc. USA) were used to assess the facial skin of the individuals. A skin age of the specific object was obtained by the image and the measuring system. When the measured skin age is higher than the actual age of the subject, they are called “bad ager”. When the measured skin age is lower than the actual age of the subject, they are called “good ager”.

### (b) Classification by Microbiome Analysis

Two methods, species relative abundance, and MNI according to the present invention were compared to classify the subjects in the test group. A “good ager” benchmark group (35 subjects) and 3 independent test groups (26 subjects each) with different product interventions were selected for discrimination analysis. Subjects in each group were recruited and given a commercial facial cleanser followed by a commercial facial cream to use twice a day for four weeks. Microbial samples were taken at baseline (0 week) and four weeks after product usage for test groups. The species relative abundance for each individual subject and MNIs for benchmark group and test groups were obtained by following same procedure as described in Example 1. The discrimination analysis (by JMP 14) was employed to compare the classification sensitivity of species relative abundance and MNI. The classification for each subject were conducted by comparing the species relative abundance (or MNI) in test group and benchmark group and be identified “bad ager” or “good ager”. If the classification is contradicted to the classification by image analysis, it is considered as misclassification.

The results for discrimination analysis were shown in Table 2.

TABLE 2

Misclassified percentage (%)

| Product Intervention | Species relative abundance | MNI |
|---|---|---|
| 1 | 5.7 | 0 |
| 2 | 3.4 | 0 |
| 3 | 6.9 | 0 |

As indicated in Table 2, MNI is more sensitive and accurate method to reflect the real condition of the skin than the species relative abundance.

1. A method of assessing skin comprising the steps of:
A) identifying a first individual microbiome network of a desired skin surface of a person in need of skin assessment;
B) comparing the first individual microbiome network with a benchmark individual microbiome network; and
C) providing a cosmetic composition to change the first individual microbiome network toward the benchmark individual microbiome network, wherein identifying a first individual microbiome network comprises the steps of:
(a) obtaining a first population microbiome network from a group of samples consisting of N individuals including said person, wherein N is an integer of at least 20; (b) obtaining a second populational microbiome network from the group of samples without the sample from said person; and
(c) determining the first individual microbiome network of said person by subtracting effects of the second population microbiome network from the first population microbiome network.

2. The method according to claim 1 wherein obtaining a population microbiome network comprises the step of determining microbiome composition data of the desired skin surface; and generating a population microbiome network by representing microbial organisms in each matrix as a network of plurality of nodes corresponding to the group of samples.

3. The method according to claim 2 wherein the microbiome composition comprises a set of taxa comprising at least one of: Marvinbryantia (genus), Erysipelotrichales (order), Erysipelotrichia (class), Bacteroidetes (phylum), Staphylococcus (genus), Staphylococcaceae (family), Bacillales (order), Actinobacteria (class), Firmicutes (phylum), Actinobacteria (phylum), and Propionibacterium (genus).

4. The method according to claim 1 wherein step (B) comprises identifying a first shift of the first individual microbiome network toward the benchmark individual microbiome network.

5. The method according to claim 4 wherein the method further comprises a step of assessing the skin conditions of the desired skin surface and associating the first shift with the skin conditions.

6. The method according to claim 5 wherein the method further comprises a step of assessing the skin conditions of the desired skin surface and associating the first shift with the skin conditions.

7. The method according to claim 1 wherein the benchmark individual microbiome network is obtained by selecting a group of benchmark persons and obtaining an averaged benchmark individual microbiome network from the benchmark skin surface of the group of benchmark persons; or finding a benchmark individual microbiome network from a first database.

8. The method according to claim 1 wherein the method comprises the step of repeating (A), (B), and (C) until the identified first microbiome network is substantially the same as the benchmark individual microbiome network.

9. The method according to claim 1 wherein the cosmetic composition comprises pigment, moisturizing agent, organic sunscreen, skin glowing agent, fragrance, natural extract, or a combination thereof.
